(19) United States
(12) Patent Application Publication
(10) Pub. No.: US 2004/0229340 A1
(43) Pub. Date: Nov. 18, 2004

(54) Method of culturing bacterium cell belonging to enterococcus genus and method of producing killed bacterium cell belonging to enterococcus genus

(76) Inventor: Yasuo Kawai, Ito-shi (JP)

Correspondence Address: HAUPTMAN KANESAKA BERNER PATENT AGENTS, LLP, Suite 300, 1700 Diagonal Road, Alexandria, VA, 22314, US

(21) Appl. No.: 10/844,376

(22) Filed: May 13, 2004

(30) Foreign Application Priority Data
May 15, 2003 (JP) 2003-136673

(51) Int. Cl. C12N 1/20

(52) U.S. Cl. 435/252.3; 435/252.8

(57) ABSTRACT

To provide a method of culturing which keeps physiology activity of bacterium cell belonging to enterococcus genus and method of producing killed bacterium cell belonging to enterococcus genus.

The method of culturing bacterium cells belonging to enterococcus genus, the method cultures bacterium cells belonging to enterococcus genus in an aerobic manner under the culturing conditions of (i) pH: 5.5 to 7.5, (ii) stirring method: 5 to 40 times per minute, (iii) temperature conditions: 30 to 40° C., and (iv) culturing time: 6 to 24 hours, and extracts viable bacteria from culture solution.

### BACKGROUND OF THE INVENTION

1. Field of the Invention

The present invention relates to a method of culturing bacterium cells belonging to the enterococcus genus and a method of producing killed bacterium cells belonging to the enterococcus genus.

2. Description of Prior Art

With the recent increase of consumption of animal fat per Japanese, and the progress towards Europe and America of the meal life, the number of lifestyle diseases, hyperlipidemia which is one of adult ailments, and patients of atherosclerosis is increasing.

Therefore, a variety of medical drugs for the hyperlipidemia have been put to practical use. However, it has been reported that all of these drugs used for suppressing the diseases have side effects such as discomfort of a stomach, vomiting, abnormal function of the liver, acute insufficiency of kidney, a feeling of lassitude, or the like after taking, and further, sometimes, persons who are taking other drugs may have to stop taking them.

The inventor of the present application discovered as a result of study of a new anti-atherosclerosis agent that bacterium cells, particularly viable bacteria and killed bacterium cells of a variety of microorganisms belonging to the enterococcus genus cause the blood cholesterol value and the triglyceride value to reduce remarkably, and obtained knowledge that since the origin of the microorganisms is intestinal bacteria which are originally present internally of the human body, even if taking it, there is nonpoisonous, and there is no side effects like the conventional anti-atherosclerosis agent or the medical drugs for the hyperlipidemia (for example, Japanese Patent No. 2756778).

Further, the inventor showed as one of knowledge that these microorganisms have a variety of physiology activity, however, these have the activity for causing the cholesterol value and the triglyceride value to reduce remarkably as one example of physiology activity thereof, not only in their viable bacteria but also in killed bacterium cell processed under the fixed conditions.

### SUMMARY OF THE INVENTION

However, thus, the inventor showed that even if it is the bacterium cells belonging to enterococcus genus having the activity reducing the cholesterol value and the triglyceride value remarkably, the activity of the bacterium cells reduced and the effect expected is not obtained depending on the method of culturing. Further, the killed bacterium cells belonging to the enterococcus genus are also known to reduce the cholesterol value and the triglyceride value remarkably, however, it showed that the effect expected is lost depending on the method of processing getting the killed bacterium cells from the vialble bacteria.

The present invention relates to a method of culturing which makes no loss of the activity of bacterium cells belonging to enterococcus genus having the effect to reduce blood cholesterol and triglyceride, and a method of producing killed bacterium cells belonging to the enterococcus genus.

Therefore, the present application provides a method of culturing bacterium cells belonging to the enterococcus genus, having the activity reducing cholesterol and triglyceride, the method comprising: culturing the enterococcus genus in an aerobic manner under the culturing conditions of (i) pH: 5.5 to 7.5, (ii) stirring method: 5 to 40 times per minute, (iii) temperature conditions: 30 to 40° C., and (iv) culturing time: 6 to 24 hours, and extracting viable bacteria from the culture solution.

Here, the culture medium composition in the culturing contains (i) glucose: 1 to 4%, (ii) yeast extract: 3 to 6%, (iii) pepton: 0.1 to 5%, and (iv) potassium phosphate, dinobasic (K₂HPO₄): 1 to 5%.

Incidentally, the enterococcus genus are one or more than two kinds of viable bacteria from a group comprising enterococcus faecalis, enterococcus faecium, enterococcus bovis, enterococcus avium, enterococcus durans, enterococcus salivarius, enterococcus mitis, and enterococcus equines.

The present invention further provides a method of producing killed bacterium cells belonging to the enterococcus genus, having the activity reducing the cholesterol and the triglyceride by (1) culturing said bacterium cells in an aerobic manner under the culturing conditions of (i) pH: 5.5 to 7.5, (ii) stirring method: 5 to 40 times per minute, (iii) temperature conditions: 30 to 40° C., and (iv) culturing time: 6 to 24 hours; (2) washing a bacterium liquid containing said viable bacteria with physiological sodium chloride water; and (3) leaving a bacterium liquid obtained by suspending it in a physiological sodium chloride aqueous solution under the environments (i) temperature: 80 to 125° C., (ii) pressure: 5 to 25 pounds, and (iii) time: 5 to 25 minutes.

Here, the culture medium composition in said (1) culturing contains (i) glucose: 1 to 4%, (ii) yeast extract: 3 to 6%, (iii) peptone: 0.1 to 5%, and (iv) potassium phosphate, dinobasic (K₂HPO₄): 1 to 5%.

### BEST MODE FOR CARRING OUT OF THE INVENITION

In the following, the kind of microorganisms that can be used in the method of culturing bacterium cells belonging to the enterococcus genus and the method of producing killed bacterium cells belonging to the enterococcus genus according to the present invention, the culturing method for the microorganisms, the method of producing killed bacterium cells of the cultured microorganisms and the medical action of bacterium cells and killed bacterium cells obtained by the result will be described in detail.

1. Microorganisms

The enterococcus genus are used, and particularly, there are illustrated, as suitable material, one or more than two kinds of the viable bacteria from a group comprising enterococcus faecalis, enterococcus faecium, enterococcus bovis, enterococcus avium, enterococcus durans, enterococcus salivarius, enterococcus mitis, and enterococcus equines. Further, the bacterium cells belonging to the above-described enterococcus genus can obtain, for example, as a deposition number ┌ATCC700802┐ in ATCC (American Type Culture Collection).

2. Conditions For Producing the Viable Bacteria

The conditions for culturing the viable bacteria are as follows:

(i) Hydrogen ion exponent (pH): the range from 5.5 to 7.5.

(ii) Stirring method: gently stir 5 to 40 times per minute.

(iii) Culturing time: the range from 6 to 24 hours.

(iv) Culture medium compositions: glucose; 1 to 4%, yeast extract; 0.3 to 6%, peptone; 0.1 to 5%, and potassium phosphate, dinobasic (K₂HPO₄); 1 to 5%.

(v) Temperature: under the temperature conditions of 30 to 45° C.

(vi) It is considered that aerobic culturing is carried out, but addition of enzyme is not particularly necessary.

3. Conditions For Producing the Killed Bacterium Cells

The viable bacterium culture solution obtained on the basis of the vital bacteria producing conditions described in the above 2 is further washed about one to two times with physiological brine, and then suspended in physiological salt aqueous solution to obtain a bacterium liquid, which liquid is subjected to heating/pressure processing under the conditions mentioned below.

(i) Temperature: 80 to 125° C.

(ii) Pressure: 5 to 25 pounds

(iii) Time: 5 to 25 minutes

The actively killed bacterium cells of said microorganisms can be obtained by the heating/high pressure processing in accordance with the aforementioned conditions.

4. Medical Action

As shown in Experimental Examples described below, it shows by the method of producing killed bacterium cells according to the present invention, the enterococcus genus causes the blood cholesterol and triglyceride values to reduce extremely effectively.

### (1) EXPERIMENTAL EXAMPLE 1

The viable bacteria of various microorganisms belonging to the enterococcus genus shown in the above 1 and the heating/pressing processed bacteria were orally administered, in the amount corresponding to 10¹⁰/day of viable bacteria and 10¹⁰/day of killed bacterium cells, every day for four weeks to mice (male, 18 weeks old, average weight 20 g: 10 mice in each group) to measure the reducing rate of cholesterol in a serum. The results of this experiment are shown in Table 1.

TABLE 1
Comparison of the cholesterol reducing rate between viable
cells and killed cells (mouse)

| Kind of microorganisms | Viable cells (%) | Killed cells (%) |
|---|---|---|
| E. faecalis K-23 | 19.5 | 44.0 |
| E. faecalis K-10 | 17.5 | 32.2 |
| E. faecalis K-57 | 18.3 | 30.4 |
| E. faecium K-58 | 19.0 | 32.5 |
| E. avium K-56 | 16.4 | 28.7 |
| E. avium K-57 | 15.8 | 23.6 |
| E. avium K-59 | 16.5 | 27.3 |
| E. durans K-66 | 12.7 | 22.8 |
| E. durans K-67 | 13.2 | 21.0 |
| E. bovis K-76 | 14.6 | 22.5 |

### (2) EXPERIMENTAL EXAMPLE 2

Similarly to the above-described Experimental Example 1, the viable bacteria of various microorganisms belonging to the enterococcus genus and the heating/pressing processed killed bacterium cells were orally administered, in the amount corresponding to 10¹⁰/day of viable bacteria and 10¹⁰/day of killed bacterium cells, every day for four weeks to normal rats (male, 18 weeks old, average weight 250 g: 7 rats in each group) to measure the reducing rate of cholesterol in a serum. The results of this experiment are shown in Table 2.

TABLE 2

| | Viable | Killed cells (%) | | |
|---|---|---|---|---|
| | cells (%) | | Normal rat | Normal rat |
| Kind of microorganisms | Normal rat | Normal rat | (*1) | (*2) |
| E. faecalis K-23 | 13.0 | 28.1 | 38.1 | 29.3 |
| E. faecalis K-10 | 11.0 | 20.0 | 22.8 | 18.2 |
| E. faecalis K-57 | 11.4 | 19.6 | 21.4 | 19.0 |
| E. faecium K-58 | 9.6 | 17.5 | 17.6 | 15.3 |
| E. avium K-56 | 8.4 | 16.7 | 17.2 | 14.8 |
| E. avium K-57 | 8.2 | 16.5 | 16.8 | 14.5 |
| E. avium K-59 | 8.2 | 17.3 | 17.5 | 13.3 |
| E. durans K-66 | 9.5 | 17.7 | 18.0 | 15.1 |
| E. durans K-67 | 9.4 | 17.0 | 17.8 | 14.7 |
| E. bovis K-76 | 9.8 | 17.5 | 18.2 | 15.2 |

(*1) Cholesterol load diet rat
(*2) Fructose load diet rat

### (3) EXPERIMENTAL EXAMPLE 3

The viable bacteria of various microorganisms belonging to the enterococcus genus and the heating/pressing processed killed bacterium cells, regulated in accordance with the above-described viable bacteria regulating example and killed bacterium cell regulating example, were orally administered, in the amount corresponding to 10¹⁰/day of viable bacteria and 10¹⁰/day of killed bacterium cells, every day for four weeks to normal mice (18 weeks old, average weight 20 g: 10 mice in each group) to measure the reducing rate of triglyceride in a serum. The results of this experiment are shown in Table 3.

TABLE 3

| Kind of microorganisms | Viable cells (%) | Killed cells (%) |
|---|---|---|
| E. faecalis K-23 | 17.1 | 76.1 |
| E. faecalis K-10 | 19.0 | 43.2 |
| E. faecalis K-57 | 17.3 | 37.0 |
| E. faecium K-58 | 17.3 | 66.4 |
| E. avium K-56 | 17.9 | 40.1 |
| E. avium K-57 | 15.1 | 33.2 |
| E. avium K-59 | 9.7 | 30.4 |
| E. durans K-66 | 9.8 | 55.1 |
| E. durans K-67 | 9.9 | 74.2 |
| E. bovis K-76 | 9.5 | 61.6 |

### (4) EXPERIMENTAL EXAMPLE 4

The viable bacteria of various microorganisms belonging to the enterococcus genus and the heating/pressing processed killed bacterium cells, regulated similarly to the above-described Experimental Example 3, were orally administered, in the amount corresponding to 10¹⁰/day of viable bacteria and 10¹⁰/day of killed bacterium cells, every day for four weeks to normal rats (male, 18 weeks old, average weight 250 g: 7 rats in each group) to measure the reducing rate of triglyceride in a serum. The results of this experiment are shown in Table 4.

TABLE 4

| | Viable | Killed cells (%) | | |
|---|---|---|---|---|
| | cells (%) | | Normal rat | Normal rat |
| Kind of microorganisms | Normal rat | Normal rat | (*1) | (*2) |
| E. faecalis K-23 | 34.9 | 61.7 | 54.8 | 69.1 |
| E. faecalis K-10 | 25.4 | 36.5 | 49.3 | 62.7 |
| E. faecalis K-57 | 34.5 | 44.3 | 41.6 | 47.8 |
| E. faecium K-58 | 18.5 | 43.5 | 39.6 | 46.5 |
| E. avium K-56 | 19.0 | 33.4 | 34.8 | 43.5 |
| E. avium K-57 | 21.4 | 32.6 | 36.5 | 36.4 |
| E. avium K-59 | 13.6 | 39.7 | 38.7 | 31.0 |
| E. durans K-66 | 13.3 | 37.4 | 43.9 | 48.7 |
| E. durans K-67 | 12.2 | 22.8 | 28.6 | 33.6 |
| E. bovis K-76 | 18.7 | 37.8 | 36.3 | 40.4 |

(*1) Cholesterol load diet rat
(*2) Fructose load diet rat

(5) Safety of Microorganisms Belonging to the Enterococcus Genus

In the acute toxicity test, enterococcus faecalis K-23 heating/pressing processed bacteria in the amount as much as possible were orally administered to rats (male, 18 weeks old, average weight 250 g: 10 rats in a group) (5600 mg/kg body weight), but no abnormality was recognized, and in the oral administration, in fact, there was no poisonous. Further, also in the intraperitoneal administration (male mice, 18 weeks old, weight 20 g: 10 mice in each group), there was high in value, e.g. 802 mg/kg body weight. The experimental result on the safety is shown in Table 5.

TABLE 5
Safety of enterococcus faecalis KAWAI

| | | |
|---|---|---|
| E. faecalis | Killed cells | >5600 mg/kg body weight |
| | Oral administration | |
| | Killed cells | >802 mg/kg body weight |
| | Intraperitoneral administration | |

As described above, in the method of culturing bacterium cells belonging to the enterococcus genus of the present invention, the method cultures the bacterium cells belonging to the enterococcus genus in an aerobic manner under the culturing conditions of (i) pH: 5.5 to 7.5, (ii) stirring method: 5 to 40 times per minute, (iii) temperature conditions: 30 to 40° C., and (iv) culturing time: 6 to 24 hours, and extracts viable bacteria from the culture solution.

Further, the killed bacterium cells of the bacterium cells are obtained by culturing the viable bacterium cells cultured as the above, washing a bacterium liquid containing said viable bacteria with physiological sodium chloride water and processing a bacterium liquid obtained by suspending it in a physiological sodium chloride aqueous solution under the environments (i) temperature: 80 to 125° C., (ii) pressure: 5 to 25 pounds, and (iii) time: 5 to 25 minutes.

Therefore, the present invention established the method of culturing bacterium cells belonging to the enterococcus genus having effect to reduce the blood cholesterol and the triglyceride, and the method of producing the killed bacterium cells thereof.

The present invention provides the method of culturing keeping the physiology activity of the bacterium cells belonging to the enterococcus genus and the method of producing the killed bacterium cells belonging to the enterococcus genus, and has the availability in the industrial field.

What is claimed is:

1. A method of culturing bacterium cells belonging to the enterococcus genus, the method comprising: culturing bacterium cells belonging to the enterococcus genus in an aerobic manner under the culturing conditions of (i) pH: 5.5 to 7.5, (ii) stirring method: 5 to 40 times per minute, (iii) temperature conditions: 30 to 40° C., and (iv) culturing time: 6 to 24 hours, and extracting viable bacteria from the culture solution.

2. The method of culturing according to claim 1, wherein the culture medium composition in said culturing contains (i) glucose: 1 to 4%, (ii) yeast extract: 0.3 to 6%, (iii) pepton: 0.1 to 5%, and (iv) potassium phosphate, dinobasic (K₂HPO₄): 1 to 5%.

3. The method of culturing according to claim 1, wherein said bacterium cells belonging to the enterococcus genus are one or more than two kinds of viable bacteria from a group comprising enterococcus faecalis, enterococcus faecium, enterococcus bovis, enterococcus avium, enterococcus durans, enterococcus salivarius, enterococcus mitis, and enterococcus equines.

4. A method of producing killed bacterial cells belonging to the Enterococcus genus comprising:
(1) culturing bacterial cells of the Enterococcus in an aerobic manner under the following culturing conditions:
(i) a pH of 5.5 to 7.5;
(ii) a stirring rate of 5 to 40 revolutions per minute;
(iii) a temperature of 30 to 40 degrees Centigrade; and
(iv) a culturing period of from 6 to 24 hours;
(2) washing resulting viable bacteria cells with physiological aqueous saline solution; and
(3) treating a bacterial liquid containing said viable bacterial cells suspended in a physiological aqueous saline solution under the following conditions:
(i) a temperature of from 80 to 125 degrees Centigrade;
(ii) a pressure of from 5 to 25 pounds per square inch [34.73 to 172.37 kPa]; and
(iii) a period of from 5 to 25 minutes;
thereby to kill said viable bacterial cells.

5. The method of producing a cholesterol and triglyceride reducing agent according to claim 4, wherein the culture medium composition in said (1) contains (i) glucose: 1 to 4%, (ii) yeast extract: 0.3 to 6%, (iii) pepton: 0.1 to 5%, and (iv) potassium phosphate, dinobasic (K₂HPO₄): 1 to 5%.

6. The method of producing a cholesterol and triglyceride reducing agent according to claim 4, said the enterococcus genus are one or more than two kinds of viable bacteria from a group comprising enterococcus faecalis, enterococcus faecium, enterococcus bovis, enterococcus avium, enterococcus durans, enterococcus salivarius, enterococcus mitis, and enterococcus equines.
